(19) United States
(12) Patent Application Publication
(10) Pub. No.: US 2023/0248945 A1
(43) Pub. Date: Aug. 10, 2023

(54) SEALING AND SECURING DRESSING SYSTEM FOR VASCULAR ACCESS DEVICES

(71) Applicant: One IV Solutions, LLC, Dunnellon, FL (US)

(72) Inventors: Robert E. Helm, Rye Beach, NH (US); Gregory A. Helm, Marlboro, VT (US)

(21) Appl. No.: 18/165,644

(22) Filed: Feb. 7, 2023

Related U.S. Application Data
(60) Provisional application No. 63/267,622, filed on Feb. 7, 2022.

(51) Int. Cl. A61M 25/02

(52) U.S. Cl. CPC A61M 25/02; A61M 2025/0266; A61M 2205/583

(57) ABSTRACT

Devices, systems, and methods used to seal and secure a medical device at an insertion site are disclosed. The systems include a mating dressing device, a central connector, and a compression lock collar. The mating dressing device includes an adhesive plate portion that forms a first circumferential seal around the insertion site and a mating body portion that the central connector form fits to. The compression lock collar compresses the mating body portion against the central connector to form a second circumferential seal. The systems can also include a segment of intravenous tubing coupled to the central connector and used for application of the mating dressing device and compression lock collar.

### RELATED CASES

This application claims priority to U.S. Provisional Application No. 63/267,622, filed on Feb. 7, 2022 and titled “SEALING DRESSING SYSTEM FOR VASCULAR ACCESS DEVICES,” which is hereby incorporated by reference in its entirety.

### TECHNICAL FIELD

The present invention relates to methods and devices for circumferentially sealing and securing medical devices in order to decrease complication and failure rates. Medical devices include, but are not limited to, vascular access catheters or other devices inserted into the human body.

### BRIEF DESCRIPTION OF THE DRAWINGS

The written disclosure herein describes illustrative embodiments that are non-limiting and non-exhaustive. Reference is made to certain of such illustrative embodiments that are depicted in the figures, in which:

FIG. 1 is a top view of an embodiment of a catheter dressing system.

FIG. 2 is a side view of the catheter dressing system of FIG. 1.

FIG. 3 is a bottom view of the catheter dressing system of FIG. 1.

FIG. 4 is a side cross-sectional view of the catheter dressing system of FIG. 1.

FIG. 5 is an end view of a proximal end of the catheter dressing system of FIG. 1.

FIG. 6 is a perspective view of an embodiment of a central connector of the catheter dressing system of FIG. 1.

FIG. 7 is a perspective view of a portion of another embodiment of a central connector of the catheter dressing system of FIG. 1.

FIG. 8 is a perspective view of a portion of another embodiment of a central connector of the catheter dressing system of FIG. 1.

FIG. 9 is a side cross-section view of an embodiment of a compression collar of the catheter dressing system of FIG. 1.

FIG. 10 is a partial side cross-section view of a portion of the catheter dressing system of FIG. 1 with the compression collar of FIG. 9 compressing a gasket portion of a dressing device of FIG. 1.

FIG. 11 is a side cross-section view of another embodiment of a compression collar.

FIG. 12 is a side cross-section view of the compression collar of FIG. 11 compressing the gasket portion of the dressing device of FIG. 1.

FIG. 13 is a side cross-section view of another embodiment of a compression collar.

FIG. 14 is a side cross-section view of the compression collar of FIG. 13 compressing the gasket portion of the dressing device of FIG. 1.

FIG. 15 is a side cross-section view of another embodiment of a compression collar.

FIG. 16 is a side cross-section view of the compression collar of FIG. 15 compressing the gasket portion of the dressing device of FIG. 1.

FIG. 17 is a side view of a first method of use step of the catheter dressing system of FIG. 1.

FIG. 18 is a side view of a second method of use step of the catheter dressing system of FIG. 1.

FIG. 19 is a side view of a third method of use step of the catheter dressing system of FIG. 1.

FIG. 20 is a side view of a fourth method of use step of the catheter dressing system of FIG. 1.

FIG. 21 is a top view of another embodiment of a dressing device.

### DETAILED DESCRIPTION

Peripheral, central and other (e.g., intra-osseous, umbilical, etc.) vascular access catheter insertion and care may be complex and variable processes involving gathering, setting up, and using multiple disparate pieces of equipment. This may lead to a variable and contaminated result. Deficiencies of vascular access catheter care may fall into two groups: (1) inadequate catheter protection, leading to internal and external contamination of the catheter, and (2) inadequate catheter securement and stabilization, leading to tissue injury and catheter damage/dislodgement.

In some instances, patch-type vascular access dressing technologies and application techniques may not allow clinically reproducible and durable sterile-sealing protection of the vascular access catheter. In some embodiments, the dressing technology does not seal and protect the catheter and its insertion site resulting in catheter contamination. In some circumstances, placing and preserving a sterile catheter and catheter site using an “aseptic no touch” technique have failed and have contributed to a rate of vascular catheter access failure of 35-60% for peripheral intravenous catheters (PIVC). Failure can lead to patient morbidity and mortality. In certain embodiments, the problem of contamination is compounded by inadequate catheter securement and stabilization. Lack of stabilization and securement may lead to catheter failure through inadvertent pull-out and through traumatic injury to the blood vessel and/or surrounding soft tissue.

An embodiment of the catheter dressing system presented herein can include three mating parts: (1) a central connector that through its shape, contour features, and interaction points, can serve as a central hub of the system; (2) a dressing device that can mate in form-fit fashion to the central connector and has an opening on its gasket portion or proximal wall that can allow the dressing device to serve as a circumferentially sealing gasket; and (3) a circumferential compression lock collar that can engage features of the central connector and gasket portion of the dressing device to lock these two elements into position to form a circumferential seal. When the three parts are mated together and when a skin adhesive plate component of the dressing device is attached to the skin surrounding the insertion point of the catheter or other device, circumferential seals around the central connector and the attached catheter may be formed.

In one embodiment, the central connector may include a connector device and a mounting shaft. The connector device may have a male Luer lock or other connection type feature that can mate to the hub of an inserted IV catheter or other device. The connector device can have an external surface contour feature that may form-fit to the dressing device when placed over the connector device to help stabilize, secure, and seal the IV catheter or other device as well as it's insertion site when an adhesive plate component of the mating dressing device is attached to the skin surrounding the catheter (or other inserted device) skin insertion point. The mounting shaft can extend proximally from a proximal end of the connector device and may (a) act to receive and secure an attached segment of IV tubing, (b) serve as a circumferential mounting point for the mating dressing device, (c) serve as a flat surface to receive and allow for compression of the sealing gasket portion of the mating dressing device, and (d) act as an interaction point for the locking circumferential compression device that compresses and locks into position the mating dressing to seal, stabilize, and secure the catheter.

The mounting shaft can include a stop-flange spaced a distance from the proximal end of the connector device to receive the gasket portion of the mating dressing device such that the gasket portion may be pressed against the proximal wall of the connector device in sealing engagement, with or without the compression lock collar that can also be slid down IV tubing into a final mating position past the stop-flange. The circumference of the stop-flange can increase as it proceeds distally, forming a cone over which the mating dressing can slide into a mating position. An abrupt perpendicular or greater angle decrease in diameter of the cone after its widest diameter may allow the gasket portion to move into position with tactile feedback that may serve to delineate correct position relative to the proximal wall of the connector device.

The mating dressing device can include a mating body portion and an adhesive plate portion. The mating body portion can: (1) mate to the connector device in form fit fashion in order to cover, stabilize, secure, and help seal the attached catheter, and (2) have an integrated gasket function so that when its soft material is pressed against the proximal wall of the connector device, a circumferential seal is formed (a seal that may be strengthened and locked into position by the compression lock collar). The adhesive plate portion may have an adhesive property or may include a separate adhesive material bonded to its underside that allows the mating dressing to be secured in sealing fashion to the skin surface surrounding the insertion site. This mating dressing can be pre-mounted around a section of extension tubing coupled to the central connector.

The locking circumferential compression collar can interact with a mating feature of the mounting shaft of the central connector to compress and lock the gasket portion of the mating dressing device against the proximal wall of the connector device to form a circumferential seal and stabilize and secure the inserted IV catheter or other inserted device. This locking compression lock collar can be pre-mounted over the segment of IV extension tubing.

The disclosed catheter dressing systems may be used to seal and secure medical devices that are inserted into the body of a patient. For example, the systems disclosed herein can be used to seal and secure straight peripheral intravenous (IV) catheters, integrated peripheral IV catheters, peripherally inserted central (PIC) catheters, midline catheters, central line catheters, umbilical catheters, intra-aortic balloon pumps (IABP), left ventricular assist devices (LVAD), extracorporeal membrane oxygenation (ECMO) catheters, percutaneous endoscopic gastrostomy (PEG) feeding tubes, percutaneous nephrostomy devices, epidural pain catheters, temporary pacemaker access devices, etc. and can be used across a spectrum/arena of patient care, from the battlefield or emergency medical services (EMS)/emergency room (ER) front line, to intensive care units (ICU), operating rooms, patient floors and outpatient care facilities.

In another embodiment, the mating body portion and adhesive plate portion can be formed of a soft flexible material such as foam or silicone that is molded or otherwise shaped into a form that can provide a stabilizing seal around an inserted catheter. Under this embodiment, the soft dressing material acts as its own sealing gasket when it is placed against the posterior surface of a custom-shaped Luer lock device that has been attached to the hub of an IV catheter that has been inserted into a patient. The mating dressing device can be slid down into mating contour position over the central connector and its attached inserted medical device. When the mating dressing device is mounted over the stop-flange of the mounting shaft of the central connector, compression of the gasket portion of the mating dressing device against the proximal surface of the central connector occurs forming a circumferential seal. This compression sealing can be augmented and durably locked into place by a compression lock device that compresses and locks into position the gasket portion of the mating dressing device and the central connector, creating a seal that also fully stabilizes and secures the inserted medical device. The compression lock device can be circumferentially pre-mounted over a segment of extension tubing attached to the central connector, so that when slid down the tubing toward the patient, the compression lock device engages locking members of the mounting shaft that then allows progressive compression of the gasket portion of the mating dressing device. This compression feature can consist of a screw mechanism, a snap-fit mechanism, a lock-pin mechanism, or any other mechanism that acts to lock the compression of the gasket portion into either a permanent or reversible stabilizing or sealing position.

In one embodiment, the compression lock collar that serves to compress the soft gasket portion of the mating dressing device against the proximal wall of the central connector can be attached to the mating dressing device to form one pre-connected unit that is slid down into position over the central connector.

(1) In one embodiment, the sterile sealing securing dressing and the compression lock collar can be pre-mounted around a segment of tubing. The central connector may be attached to the distal end of the tubing and a needle-free device or other connector may be attached to the proximal end of the tubing. The mating dressing device can be pre-mounted on the tubing between the two connector ends, the mating dressing device may have the ability to slide freely along the tubing through its two open circumferential orifices, both of which are in their open and non-sealed state. The pre-mounting of the dressing around the segment of tubing with its attached connectors can allow: (1) the mating dressing device and central connector can be packaged together as one sterile item in one sterile package that eliminates the need to open and assemble multiple individual components, (2) the mating dressing device can be packaged so that it is adjacent to the proximal connector, leaving the distal tubing and central connector easily available for finger-grasping and attachment to a hub of the inserted medical device, (3) once the central connector is attached to the hub and the mating dressing device slid into final mounting position around the hub-central connector complex, the mating dressing device can be grasped to stabilize the underlying catheter hub-tubing connector complex—over which it is mounted—while two circumferential seals are effected. The first circumferential seal is achieved by removing an adhesive backing from the mating dressing device and adhering the mating dressing device to the skin, and the second circumferential seal can be achieved by deploying the compression lock collar to press the gasket portion of the mating dressing device against the proximal surface of the central connector. These two sealing steps can be affected in any preferred order.

In some embodiments, the steps to use the sealing and securing dressing system presented in this disclosure can be: removing the sealing and securing dressing system from its sterile packaging; attaching the connector of the central connector to the hub of the inserted medical device; sliding the mating dressing device distally over the tubing of the central connector into mating position over the central connector-hub complex; activating the compression lock collar to compress the gasket portion of the mating dressing device against the proximal wall of the central connector and locking the compression with the compression lock collar; peeling off the release liner of the adhesive plate; and adhering the adhesive plate to the skin.

In some embodiments, a segment of extension tubing can be coupled to an inserted medical device. The disclosed dressing systems may include the extension tubing as a dressing applicator, thereby reducing the number movements back and forth between a bedside tray, where the dressing materials are gathered and arranged, and the patient's medical device insertion site.

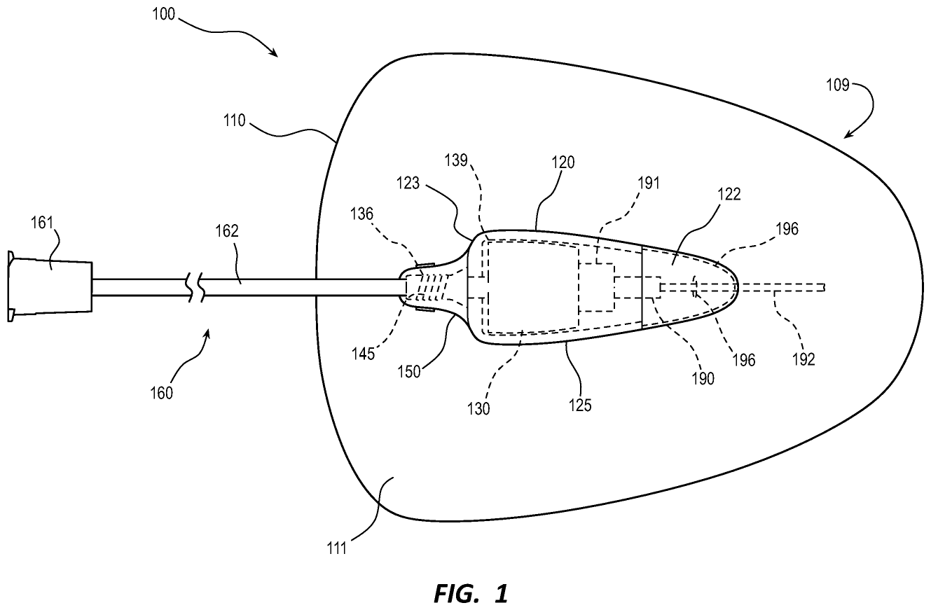

FIGS. 1-5 illustrate an embodiment of a sealing and securing catheter dressing system or catheter dressing system 100 that is configured to seal around, stabilize, and secure an inserted medical device 190, such as a peripheral IV catheter, that has been inserted through the skin of a patient. As FIG. 1 illustrates, the catheter dressing system 100 may include a mating dressing device 109, a central connector 130, and a compression lock collar 150.

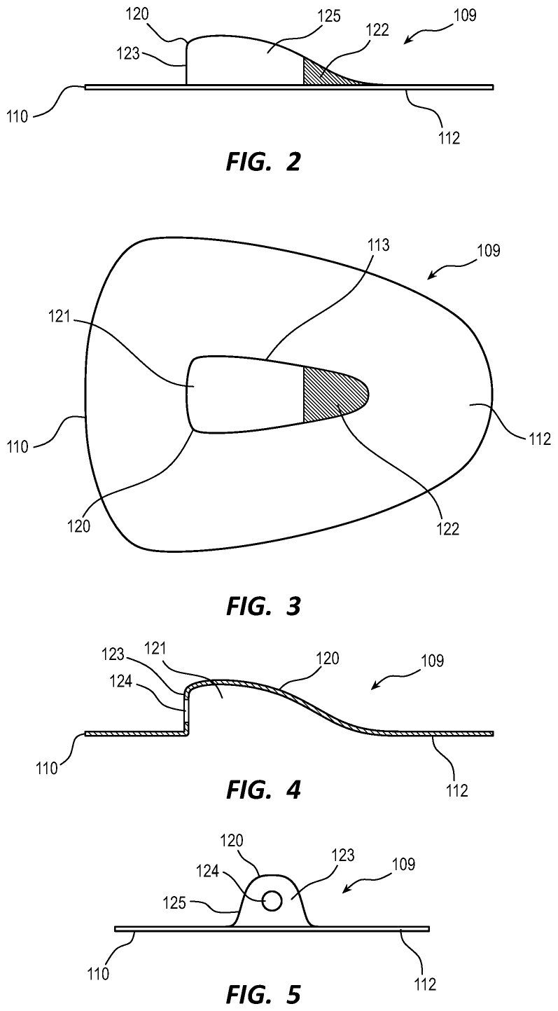

As shown in FIG. 1, the mating dressing device 109 can include an adhesive plate portion 110 and a mating body portion 120. The adhesive plate portion 110 and the mating body portion 120 may include a unibody construct where a top layer 111 of the adhesive plate portion 110 is integrally constructed with the mating body portion 120. In other words, the adhesive plate portion 110 and the mating body portion 120 can be made without joints or seams. The mating body portion 120 may extend upwardly from the adhesive plate portion 110 as shown in FIG. 2. A chamber 121 within the mating body portion 120 can be defined by side walls 125 and a substantially vertically oriented gasket portion or proximal wall 123. The chamber 121 may include a shape or contour that substantially matches an external shape or contour of at least a portion of the central connector 130. The internal dimensions of the chamber 121 can be smaller than the external dimensions of the central connector 130 to facilitate a form-fit between the mating body portion 120 and the central connector 130 when the central connector 130 is disposed within the chamber 121. The form-fit provides securement and stability to the inserted medical device 190. The chamber 121 can include a first circumferential opening 113 configured to allow placement of the central connector 130 and the inserted medical device 190 into the chamber 121. A second circumferential opening or gasket opening 124 extends through the gasket portion 123 and is configured to allow passage of a portion of the central connector 130 and mounting of the mating dressing device 109 onto the central connector 130. In certain embodiments, the unibody structure of the adhesive plate portion 110 and the mating body portion 120 can be formed by thermoforming, injection molding, or any other suitable forming technique.

In some embodiments, the top layer 111 and the mating body portion 120 may be formed from a flexible material, such as flexible foam, hydrocolloid, silicone, and polypropylene. Other suitable flexible materials may be utilized. The flexible material may allow the adhesive plate portion 110 to readily conform to a contour of a patient's body portion, such as a hand or an arm, to form a circumferential seal around an insertion site to prevent passage of water and contaminants, such as bacteria, into the chamber 121. Additionally, the flexible material may allow the mating body portion 120 to conform around the central connector 130 to seal, stabilize, and secure the inserted medical device 190. An adhesive layer 112 can be coupled or disposed on a lower surface of the top layer 111. The adhesive layer 112 can be configured to adhere to the skin of the patient. A release liner may be releasably coupled to the adhesive layer 112 until the adhesive plate portion 110 is prepared to be applied to the skin of the patient. In some embodiments, the flexible material of the top layer 111 can have adhesive properties.

The mating body portion 120 may include a window 122 configured to allow visualization of the insertion site 196 and the inserted medical device 190 through the mating body portion 120. Visualization of the insertion site 196 allows the practitioner to determine the status of the insertion site 196 and inserted medical device 190 (e.g., infection, leaking, kinking, displacement) without removal of the mating dressing device 109. The window 122 may be formed of a clear, breathable or moisture vapor permeable material that allows passage of moisture vapor through the material, such as polyurethane.

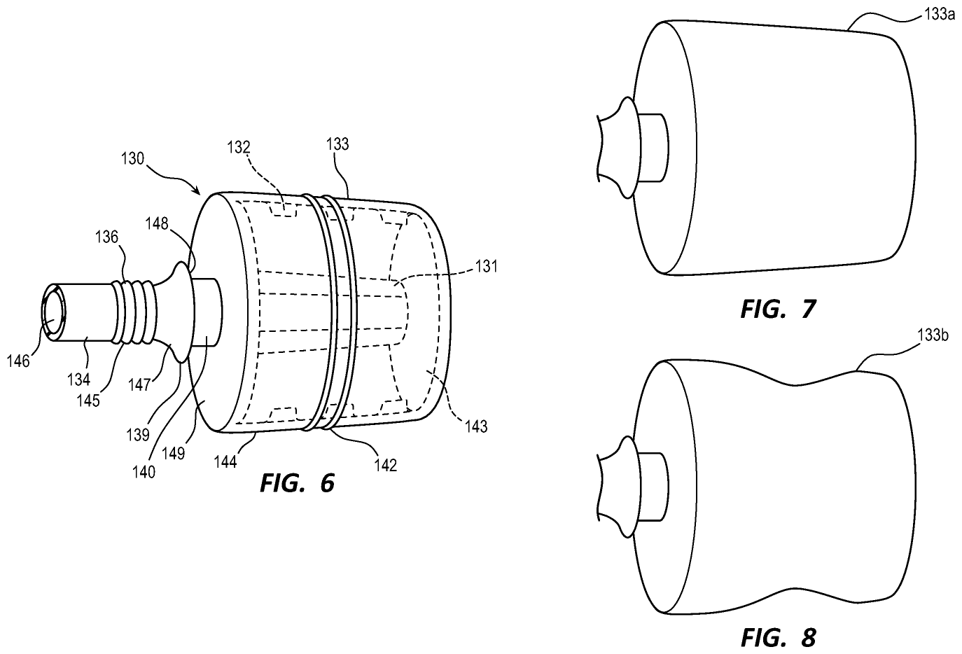

FIG. 6 illustrates an embodiment of the central connector 130. As illustrated in FIG. 6, the central connector 130 can include a barrel-shape connector device 133 and a mounting shaft 134. The central connector 130 may be formed of any suitable rigid or semi-rigid material, such as polypropylene, polyethylene, and polycarbonate. Other suitable materials may be used. The rigid or semi-rigid material can allow the central connector 130 to form-fit within the chamber 121 to stabilize the inserted medical device 190 relative to the insertion site 196. The connector device 133 may include an external gripping feature to enhance the stability (e.g., prevention of axial displacement) of the central connector 130 within the mating body portion 120. As shown in FIG. 6, the gripping feature may be one or more circumferential ridges 142 configured to engage with an internal surface of the chamber 121. FIG. 7 shows an alternative embodiment of a connector device 133a having a tapered external shape configured to prevent distal movement of the connector device 133a within the chamber 121. FIG. 8 shows another alternative embodiment of a connector device 133b having an hourglass shape configured to prevent proximal and distal movement of the connector device 133b within the chamber 121. In other embodiments, the connector device 133 may include gripping features configured to prevent rotational movement of the central connector 130. For example, the connector device 133 may include one or more longitudinal ridges configured to engage with the internal surface of the chamber 121. Other suitable gripping features to prevent rotational movement of the central connector 130 may be utilized.

As shown in FIG. 6, the connector device 133 can include a cavity 143 defined by a connector wall 144 including a proximal portion 149. The proximal portion 149 may be substantially flat and oriented substantially perpendicular to a longitudinal axis of the central connector 130. The cavity 143 may be closed at a proximal end and open at a distal end. Internal threads 132 (e.g., male Luer lock threads) may extend into the cavity 143 from the connector wall 144. The internal threads 132 can be configured to engage with external threads (e.g., female Luer lock threads) of a hub or female Luer fitting 191 (shown in FIG. 1) of the inserted medical device 190. A male fitting 131 (e.g., male Luer fitting) can be disposed within the cavity 143 and in fluid communication with the mounting shaft 134. The male fitting 131 can be configured to fluidly engage with the hub 191 when the central connector 130 is coupled to the hub 191 of the inserted medical device 190. In other embodiments, the connector device 133 and the inserted medical device 190 may include any other suitable type of compatible fittings.

The mounting shaft 134 may extend proximally from the proximal portion 149 of the connector wall 144. The mounting shaft 134 can include a bore 146 extending longitudinally through the mounting shaft 134. The bore 146 may be in fluid communication with the male fitting 131 and the hub 191. A recess 140 can be disposed between the proximal portion 149 of the connector wall 144 and a stop-flange 139. An outer diameter of the recess 140 can be sized to interfere with the inside surface of the gasket opening 124 of the gasket portion 123 to form a third circumferential seal around the mounting shaft 134. The outer diameter of the recess 140 may be smaller than an outer diameter of the stop-flange 139 and a length of the recess 140 can be substantially equivalent to a thickness of the gasket portion 123. The stop-flange 139 may include a tapered proximal face 147 and a distal face 148 that is substantially perpendicular to a longitudinal axis of the mounting shaft 134. This configuration facilitates passage of the gasket portion 123 distally over the stop-flange 139 and capture of the gasket portion 123 between the proximal portion 149 and the stop-flange 139. As the gasket portion 123 passes over the stop-flange 139, a tactile feedback, such as an increase in resistance and then a sudden loss of resistance, may be provided to the practitioner to indicate proper coupling of the mating dressing device 109 and central connector 130. A second circumferential seal may be formed between the gasket portion 123 and the proximal portion 149 as the gasket portion 123 is held against the proximal portion 149 by the stop-flange 139.

The mounting shaft 134 can include a collar lock member 145 disposed proximally of the stop-flange 139. The collar lock member 145 can be configured to engage with the compression lock collar 150 to hold a distal end of the compression lock collar 150 against the gasket portion 123. As shown in FIG. 1, the collar lock member 145 can include external threads 136 configured to threadingly engage with internal threads 153 of the compression lock collar 150.

As shown in FIG. 1, a proximal portion of the mounting shaft 134 can be configured for attachment of a distal portion of a tubing 162 of an extension tubing set 160. The bore 146 may be in fluid communication with the tubing 162. In one embodiment, the tubing 162 can be coupled to an internal surface of the bore 146 using a solvent or glue. In another embodiment, the tubing 162 may be coupled to an external surface of the mounting shaft 134 using a solvent or glue. A female connector 161, such as a female Luer fitting or needleless access device, can be coupled to a proximal end of the tubing 162.

As illustrated in FIG. 10, a compressible gasket or washer 141 can be disposed over the recess 140 of the mounting shaft 134 and positioned adjacent to the proximal portion 149. The gasket 141 can increase the compression of the compression lock collar 150 to form the second circumferential seal.

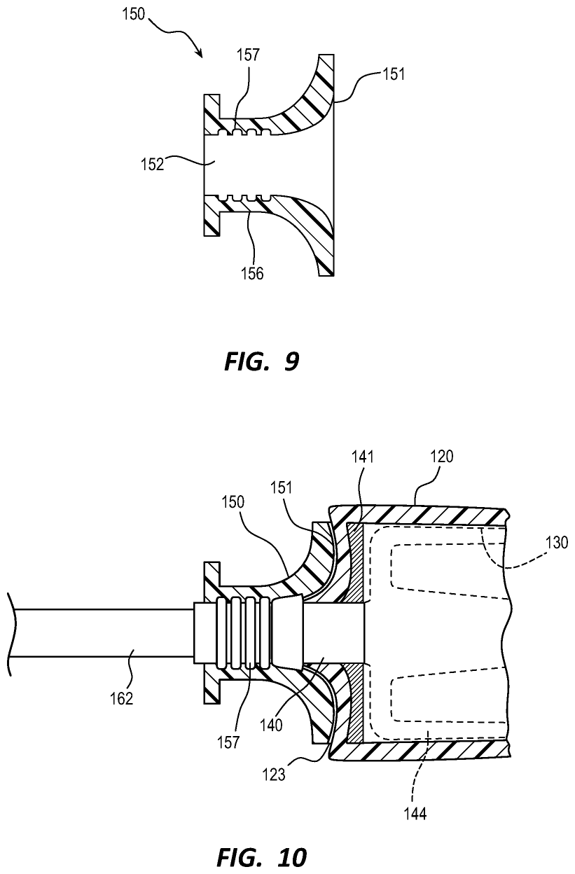

As illustrated in FIGS. 9 and 10, the compression lock collar 150 includes a body 156 defining a bore 152 configured to allow passage of the compression lock collar 150 over the tubing 162. The compression lock collar 150 may be formed from the same material or a similar material used to form the central connector 130. An engagement member 157 configured to reversibly or irreversibly engage with the collar lock member 145 of the mounting shaft 134 can be disposed within the bore 152. A distal end 151 of the compression lock collar 150 can contact and compress the gasket portion 123 of the mating body portion 120 and/or gasket 141 when the collar 150 is disposed over the mounting shaft 134. When the gasket portion 123 and/or gasket 141 is compressed by the distal end 151, a second circumferential seal between the gasket portion 123 and/or the gasket may be formed at an interface or engagement with the proximal portion 149. This second circumferential seal can prevent passage of contaminants (e.g., bacteria) through the gasket opening 124 into the chamber 121. The distal end 151 can be flared outwardly to provide a wide surface to contact and apply a compressive force to the gasket portion 123. In certain embodiments, the compression lock collar 150 is disposed over the tubing 162 separate from the central connector 130 and then displaced distally to couple with the central connector 130 following displacement of the mating dressing device 109 over the central connector 130. In another embodiment, the compression lock collar 150 is coupled to the central connector 130 and displaced distally with the mating dressing device 109 to couple with the central connector 130.

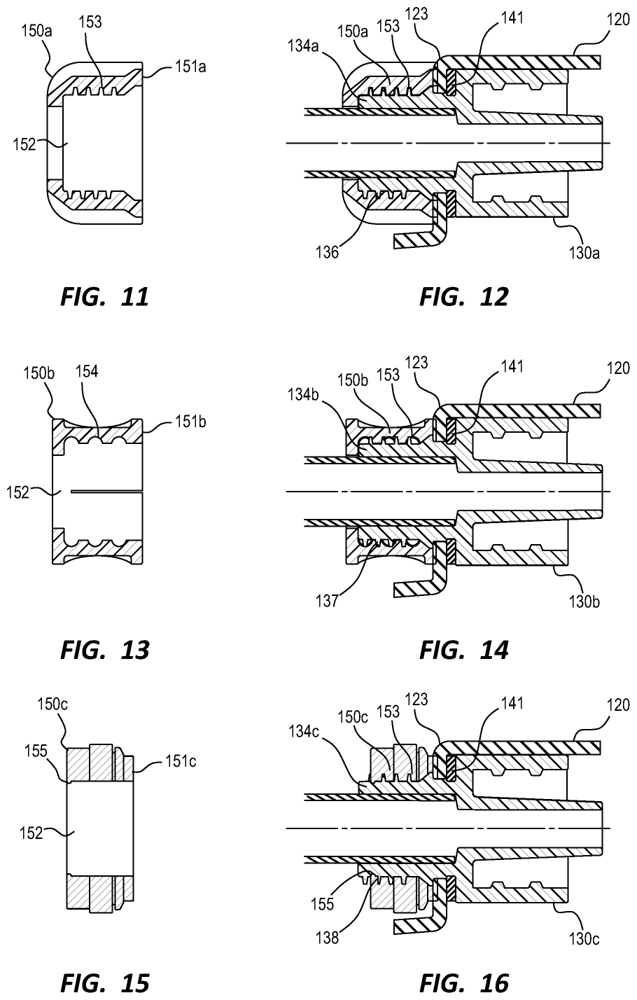

FIGS. 11 and 12 illustrate an embodiment of a compression lock collar 150a having internal threads 153 configured to engage with the external threads 136 of the mounting shaft 134a to axially lock a distal end 151a of the compression lock collar 150a against the gasket portion 123. FIGS. 13 and 14 illustrate an embodiment of a compression lock collar 150b having circumferential grooves 154 configured to engage with external rings 137 of the mounting shaft 134b to axially lock the distal end 151b of the compression lock collar 150b against the gasket portion 123. FIGS. 15 and 16 illustrate an embodiment of a compression lock collar 150c having a snap ring 155 configured to engage with external rings 138 of the mounting shaft 134 to axially lock a distal end 151c of the compression lock collar 150c against the gasket portion 123.

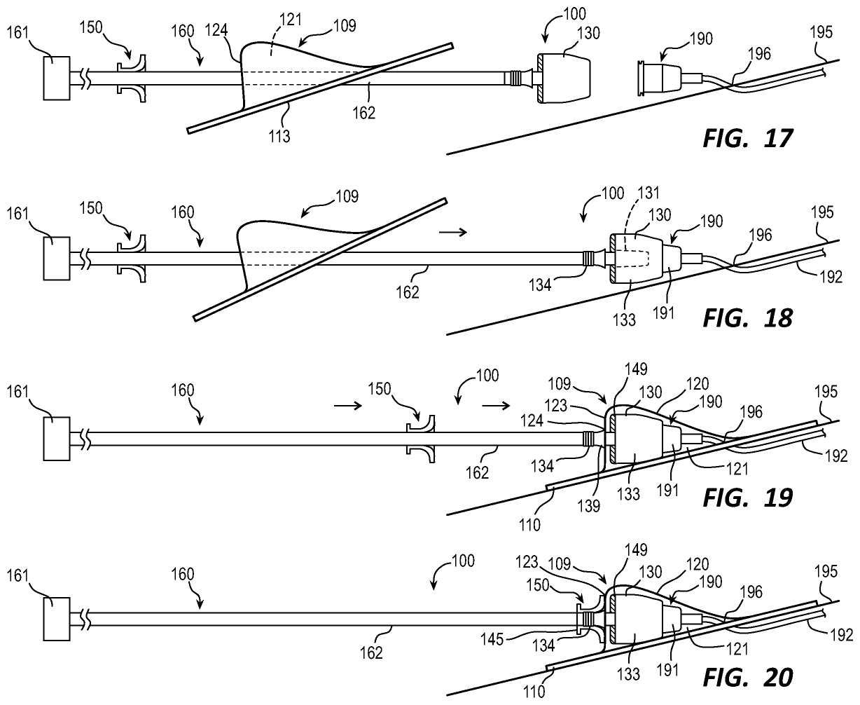

FIGS. 17-20 illustrate a method of using the catheter dressing system 100 to dress an insertion site 196 of an inserted medical device 190. When using the catheter dressing system 100 the number of individual steps may be six or less. As shown in FIG. 17, the inserted medical device 190 can be inserted through the skin of a patient at the insertion site 196 at any suitable location to provide access to the patient's vascular system. For example, the inserted medical device 190 may be inserted into a peripheral vessel in the arm or leg or into a central vessel in the neck or chest. The inserted medical device 190 can be a peripheral IV catheter; a midline catheter; a central venous catheter; a peripherally inserted central venous catheter; an arterial catheter; etc.

As shown in FIG. 17, the catheter dressing system 100 can be removed from a sterile package and readied for dressing of the insertion site 196 and inserted medical device 190. As shown, the mating dressing device 109 can be predisposed over the extension tubing set 160 between the central connector 130 and the compression lock collar 150 such that the tubing 162 is disposed through the first circumferential opening 113, the chamber 121, and the gasket opening 124. The central connector 130 may be disposed distally of the mating dressing device 109. The compression lock collar 150 may be disposed proximally of the mating dressing device 109 and distal to the female connector 161 of the extension tubing set 160. In certain embodiments, sterile caps coupled to the male fitting 131 of the central connector 130 and the female connector 161 of the extension tubing set 160 may be removed to prepare the catheter dressing system 100 for use.

As shown in FIG. 18, the central connector 130 can be coupled to the hub 191 of the inserted medical device 190. When coupled, the male fitting 131 sealingly couples with the hub 191 and the internal threads 132 engage with the external threads of the hub 191 to secure the central connector 130 and the hub 191 together. When the central connector 130 and the hub 191 are coupled, a fluid path extends from the patient's vessel, through the catheter tubing 192, through the hub 191, though the male fitting 131, through the mounting shaft 134, through the tubing 162, and through the female connector 161. This fluid path may allow fluid to be infused into or withdrawn from the patient without breaking any of the connections resulting in a closed system that can prevent internal contamination. The mating dressing device 109 may be displaced distally by the practitioner over the tubing 162.

As shown in FIG. 19, the mating dressing device 109 can be coupled to the central connector 130 such that the connector device 133 and the hub 191 are disposed within the chamber 121 and the mating body portion 120 of the mating dressing device 109 covers the insertion site 196. The connector device 133 may form fit within the chamber 121 and engage with the mating body portion 120 to stabilize the central connector 130 and inserted medical device 190 axially and/or rotationally relative to the mating dressing device 109. The mounting shaft 134 can be disposed through the gasket opening 124 and through the gasket 141, when the gasket 141 is present. The gasket portion 123 surrounding the gasket opening 124 may be disposed between the stop-flange 139 of the mounting shaft 134 and the proximal portion 149 to stabilize the mating dressing device 109 in place relative to the central connector 130. The gasket portion 123 can be held against the proximal portion 149 to form the second circumferential seal to prevent ingress of contaminants through the gasket opening 124 into the chamber 121. The compression lock collar 150 can be distally displaced over the tubing 162.

As shown in FIG. 20, the compression lock collar 150 can be displaced distally to couple with the collar lock member 145 of the mounting shaft 134 and the gasket portion 123 of the mating body portion 120. When coupled, the compression lock collar 150 can compress the gasket portion 123 resulting in compression of the gasket portion 123 against the proximal portion 149 and securement of the mating dressing device 109 relative to the central connector 130. The release liner (not shown) may be removed from the adhesive plate portion 110 to expose the adhesive surface and the adhesive plate portion 110 can be adhered to the patient's skin to form the first circumferential seal around the insertion site 196 to prevent ingress of contaminants into the chamber 121. Sterility of the chamber 121, the inserted medical device 190, and the insertion site 196 can be maintained by the first and second circumferential seals.

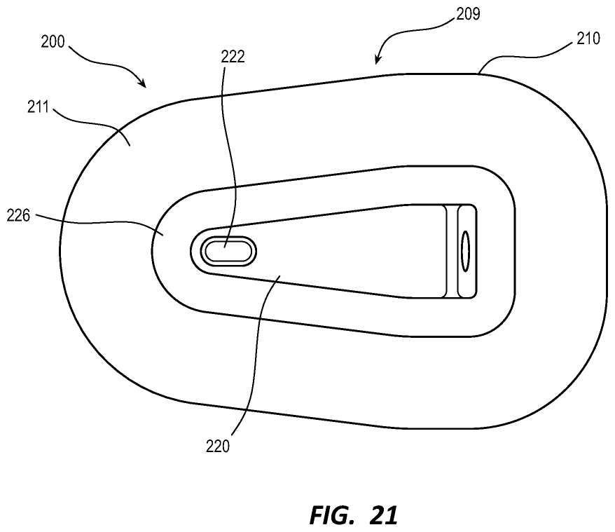

FIG. 21 depicts an embodiment of a mating dressing device 209 that resembles the mating dressing device 109 described above in certain respects. Accordingly, like features are designated with like reference numerals, with the leading digit incremented to “2.” For example, the embodiment depicted in FIG. 21 includes a mating body portion 220 that may, in some respects, resemble the mating body portion 120 of FIG. 1.

Relevant disclosure set forth above regarding similarly identified features thus may not be repeated hereafter. Moreover, specific features of the mating dressing device 109 and related components shown in FIGS. 1-20 may not be shown or identified by a reference numeral in the drawings or specifically discussed in the written description that follows. However, such features may clearly be the same, or substantially the same, as features depicted in other embodiments and/or described with respect to such embodiments. Accordingly, the relevant descriptions of such features apply equally to the features of the mating dressing device 209 and related components depicted in FIG. 21. Any suitable combination of the features, and variations of the same, described with respect to the mating dressing device 109 and related components illustrated in FIGS. 1-20 can be employed with the mating dressing device 209 and related components of FIG. 21, and vice versa. This pattern of disclosure applies equally to further embodiments depicted in subsequent figures and described hereafter, wherein the leading digits may be further incremented.

As shown in FIG. 21, the mating dressing device 209 can include an adhesive plate portion 210 and a mating body portion 220. The dressing system 200 may differ from the catheter dressing system 100 in at least the adhesive plate portion 210 and the mating body portion 220 can be separately formed components of the mating dressing device 209. The mating body portion 220 may include a flange 226 extending outwardly from a base of the mating body portion 220. When the adhesive plate portion 210 and the mating body portion 220 are assembled together, the flange 226 can be coupled to a top surface of the adhesive plate portion 210 around a first circumferential opening 213. In another embodiment, the flange may be coupled to a bottom surface of the adhesive plate portion 210 such that the mating body portion 220 is disposed through the first circumferential opening 213 and extends upwardly above the adhesive plate portion 210. The flange 226 can be coupled to the adhesive plate portion 210 using any suitable technique, such as bonding, welding, and gluing. Other coupling techniques may be suitably utilized.

Any methods disclosed herein comprise one or more steps or actions for performing the described method. The method steps and/or actions may be interchanged with one another. In other words, unless a specific order of steps or actions is required for proper operation of the embodiment, the order and/or use of specific steps and/or actions may be modified. For example, a method of placing a circumferential sealing dressing, may include fluidly coupling a central connector to a medical device inserted into a patient at an insertion site; displacing a catheter dressing device distally over an intravenous tubing segment to couple with the central connector; forming a first circumferential seal around the insertion site with an adhesive plate of the catheter dressing device; and forming a second circumferential seal at an interface of the catheter dressing device and the central connector. Other steps are also contemplated.

Embodiments may be understood by reference to the drawings, wherein like parts are designated by like numerals throughout. It will be readily understood by one of ordinary skill in the art having the benefit of this disclosure that the components of the embodiments, as generally described and illustrated in the figures herein, could be arranged and designed in a wide variety of different configurations. Thus, the following more detailed description of various embodiments, as represented in the figures, is not intended to limit the scope of the disclosure, but is merely representative of various embodiments. While the various aspects of the embodiments are presented in drawings, the drawings are not necessarily drawn to scale unless specifically indicated.

Reference throughout this specification to “an embodiment” or “the embodiment” means that a particular feature, structure, or characteristic described in connection with that embodiment is included in at least one embodiment. Thus, the quoted phrases, or variations thereof, as recited throughout this specification are not necessarily all referring to the same embodiment.

Similarly, in the above description of embodiments, various features are sometimes grouped together in a single embodiment, figure, or description thereof for the purpose of streamlining the disclosure. This method of disclosure, however, is not to be interpreted as reflecting an intention that any claim requires more features than those expressly recited in that claim. Rather, as the following claims reflect, inventive aspects lie in a combination of fewer than all features of any single foregoing disclosed embodiment.

The phrase “coupled to” is broad enough to refer to any suitable coupling or other form of interaction between two or more entities, including mechanical, fluidic and thermal interaction. Thus, two components may be coupled to each other even though they are not in direct contact with each other. The phrases “attached to” or “attached directly to” refer to interaction between two or more entities which are in direct contact with each other and/or are separated from each other only by a fastener of any suitable variety (e.g., mounting hardware or an adhesive). The phrase “fluid communication” is used in its ordinary sense, and is broad enough to refer to arrangements in which a fluid (e.g., a gas or a liquid) can flow from one element to another element when the elements are in fluid communication with each other.

The directional terms “distal” and “proximal” are given their ordinary meaning in the art. That is, the distal end of a medical device means the end of the device furthest from the practitioner during use. The proximal end refers to the opposite end, or the end nearest to the practitioner during use.

“Fluid” is used in its broadest sense, to refer to any fluid, including both liquids and gases as well as solutions, compounds, suspensions, etc., which generally behave as fluids.

References to approximations are made throughout this specification, such as by use of the term “substantially.” For each such reference, it is to be understood that, in some embodiments, the value, feature, or characteristic may be specified without approximation. For example, where qualifiers such as “about” and “substantially” are used, these terms include within their scope the qualified words in the absence of their qualifiers. For example, where the term “substantially perpendicular” is recited with respect to a feature, it is understood that in further embodiments, the feature can have a precisely perpendicular configuration.

The terms “a” and “an” can be described as one, but not limited to one. For example, although the disclosure may recite a housing having “a stopper,” the disclosure also contemplates that the housing can have two or more stoppers.

It will be apparent to those having skill in the art that changes may be made to the details of the above-described embodiments without departing from the underlying principles of the invention. Embodiments of the invention in which an exclusive property or privilege is claimed are defined as follows.

The claims following this written disclosure are hereby expressly incorporated into the present written disclosure, with each claim standing on its own as a separate embodiment. This disclosure includes all permutations of the independent claims with their dependent claims. Moreover, additional embodiments capable of derivation from the independent and dependent claims that follow are also expressly incorporated into the present written description.

Without further elaboration, it is believed that one skilled in the art can use the preceding description to utilize the invention to its fullest extent. The claims and embodiments disclosed herein are to be construed as merely illustrative and exemplary, and not a limitation of the scope of the present disclosure in any way. It will be apparent to those having ordinary skill in the art, with the aid of the present disclosure that changes may be made to the details of the above-described embodiments without departing from the underlying principles of the disclosure herein. In other words, various modifications and improvements of the embodiments specifically disclosed in the description above are within the scope of the appended claims. Moreover, the order of the steps or actions of the methods disclosed herein may be changed by those skilled in the art without departing from the scope of the present disclosure. In other words, unless a specific order of steps or actions is required for proper operation of the embodiment, the order or use of specific steps or actions may be modified. The scope of the invention is therefore defined by the following claims and their equivalents.

1. A catheter dressing system comprising:
a dressing device, comprising:
an adhesive plate portion, comprising:
a top layer of a conformable material; and
a first circumferential seal opening disposed through the top layer; and
a mating body portion coupled to the adhesive plate portion, comprising:
a gasket portion, comprising a second circumferential seal opening disposed through the gasket portion; and
a chamber comprising an internal surface; and
a central connector, comprising:
a barrel member comprising a proximal end and configured to be disposed within the chamber through the first circumferential seal opening;
a male connector configured to fluidly couple to a hub of an inserted medical device; and
a mounting shaft extending proximally from the barrel member and configured to be disposed through the second circumferential seal opening.

2. The catheter dressing system of claim 1, wherein the adhesive plate portion is configured to provide a first circumferential seal around the first circumferential seal opening when the adhesive plate portion is applied to a patient's skin.

3. The catheter dressing system of claim 1, wherein the gasket portion and the proximal end are configured to engage when the central connector is disposed within the chamber to provide a second circumferential seal around the second circumferential seal opening.

4. The catheter dressing system of claim 1, wherein the adhesive plate portion and the mating body portion comprise a unibody construct.

5. The catheter dressing system of claim 1, wherein the mating body portion comprises a flange configured to couple the mating body portion to the adhesive plate portion.

6. The catheter dressing system of claim 1, wherein the mating body portion comprises a window section configured to allow visualization of a catheter insertion site.

7. The catheter dressing system of claim 6, wherein the window comprises a moisture vapor permeable material.

8. The catheter dressing system of claim 1, wherein the adhesive plate portion further comprises an adhesive layer disposed on a bottom surface of the top layer.

9. The catheter dressing system of claim 1, wherein the barrel member comprises an external contour configured to match an internal contour of the chamber.

10. The catheter dressing system of claim 1, wherein the mounting shaft comprises a stop flange configured to capture the gasket portion between the stop flange and the proximal end.

11. The catheter dressing system of claim 1, further comprising a compression collar engagable with the mounting shaft and configured to apply a compressive force to the gasket portion.

12. The catheter dressing system of claim 1, further comprising an extension tubing set comprising:
a tubing coupled to the mounting shaft at a distal end of the tubing; and
a connector coupled to a proximal end of the tubing.

13. The catheter dressing system of claim 1, further comprising a gasket disposed between the gasket portion and the proximal end and over the mounting shaft.

14. A catheter dressing device, comprising:
a first circumferential seal opening disposed though an adhesive plate portion;
and a second circumferential seal opening disposed through a proximal wall of a mating body portion; and
a chamber in communication with the first circumferential opening and the second circumferential seal opening.

15. The catheter dressing device of claim 14, wherein the adhesive plate portion is configured to provide a first circumferential seal around the first circumferential seal opening when the adhesive plate is applied to a patient's skin.

16. The catheter dressing device of claim 14 wherein the proximal wall is configured to provide a second circumferential seal around the second circumferential seal opening when the dressing is mated with a central connector.

17. A method of placing a circumferential sealing dressing, comprising:
fluidly coupling a central connector to a medical device inserted into a patient at an insertion site;
displacing a catheter dressing device distally over an intravenous tubing segment to couple with the central connector;
forming a first circumferential seal around the insertion site with an adhesive plate of the catheter dressing device; and
forming a second circumferential seal at an interface of the catheter dressing device and the central connector.

18. The method of claim 17, further comprising mating the central connector with a chamber of the catheter dressing device.

19. The method of claim 17, further comprising engaging a compression collar to the central connector to apply a compressive force to the catheter dressing device and the central connector.

20. The method of claim 17, further comprising mounting the catheter dressing device onto the intravenous tubing segment.
